(19) United States
(12) Patent Application Publication
(10) Pub. No.: US 2001/0055797 A1
(43) Pub. Date: Dec. 27, 2001

(54) Sol-gel biomaterial immobilization

(76) Inventors: John F.T. Conroy, Alexandria, VA (US); Pamela M. Norris, Charlottesville, VA (US); Mary E. Power, Charlottesville, VA (US)

Correspondence Address: Pamela M. Norris, 1509 Still Meadow Cove, Charlottesville, VA, 22901, US

(21) Appl. No.: 09/785,188

(22) Filed: Feb. 20, 2001

Related U.S. Application Data
(60) Provisional application No. 60/183,095, filed on Feb. 17, 2000.

(51) Int. Cl. C12N 11/02; C12N 11/04

(52) U.S. Cl. 435/177; 435/182

(57) ABSTRACT

A sol and a method for forming a sol that can be used to immobilize biological materials and/or form robust macroporous gels. As needed, sols that are compatible with biological materials can be produced. Also as needed, robust macroporous gels can be formed by introducing dispersants to suitable sols.

### CROSS-REFERENCE TO RELATED APPLICATIONS

This application claims the benefit of U.S. Provisional Application No.: 60/183,095, filed Feb. 17, 2000.

### BACKGROUND OF THE INVENTION

1. Field of the Invention:

This invention is directed toward a method for forming sols and the sols themselves that can be used for the immobilization of biological materials and/or the formation of robust, macroporous samples.

The present invention includes use of various technologies referenced and described in the references identified in the appended LIST OF REFERENCES and cross-referenced throughout the specification by boldface numerals in brackets corresponding to the respective references, the entire contents of all of which are incorporated herein by reference.

2. Discussion of the Background:

Sol-gel-derived materials such as silica have been proposed both as cytocompatible scaffolds for the immobilization of cells, as well as robust, easily engineered ceramic matrices for the immobilization of biopolymers. There are several favorable characteristics of sol-gel-derived materials as immobilization matrices, including a low temperature production routes, chemical-, temperature-, and radiation-stability, high surface area and porosity, ease of functionalization, mechanical rigidity (little or no swelling), and tunable properties and microstructures.

Furthermore, in regard to the immobilization of cells, the biocompatibility of both sol-gel-derived and non-sol-gel-derived ceramic materials has already been extensively investigated. [1][2][3][4][5][6] Perhaps for this reason, the immobilization of cells within sol-gel-derived silica matrices has been the subject of several investigations. In the late 1970's, Hino et al. described a two-step synthetic route for immobilizing microorganisms within complex gels containing significant organic and sol-gel-derived silica fractions.[11] The weight percent of silica in these gels was relatively low and the gels themselves often displayed behavior characteristic of organic polymer gels, such as significant swelling in different solvents and solubility in water. In the 1980's, Carturan et al. immobilized S. cerevisiae in a multilayered sol-gel-derived thin film.[8] In the 1990's, Uo et al. pre-gelled macroporous silica matrices in solutions containing water-soluble polymers, rinsed the gelation solution from the gels, and then loaded the resulting macroporous samples with Saccharomyces cerevisiae and showed germination. [7] Pope et al. used a polymer-free, two-step synthetic route to immobilize both S. cerevisiae [9][12][13][14] and pancreatic islet cells [15][16] by adding the cells to a hydrolyzed silica solution prior to gelation. He has also developed a method for making cell-containing, sol-gel-derived silica microspheres.[17] In a similar production route, Livage et al. mixed Leishmania donovani infantum into hydrolyzed methoxysilicate sols that were later gelled and optically probed.[18] Al-Saraj et al. immobilized S. cerevisiae in silica gels derived from tetraethoxysilicate and examined the bioaccumulation of heavy metals.[10] Branyik et al. encapsulated both yeast (Candida tropicalis) and bacteria (Pseudomonas) inside similar silica gels and investigated their use in bioremediation.[19] They found that the sol-gel encapsulation procedure was relatively benign, but that the organisms were so constrained by the silica matrix that they were unable to colonize the scaffold. Rietti-Shati et al. also immobilized a strain of Pseudomonas within silica gels using both pure sol-gel-derived silica and combined silica/Ca-alginate system. They obtained best results with the pure sol-gel-derived silica system. An alternative to encapsulation in a liquid sol is termed Biosil and involves cell immobilization upon fiber supports followed by exposure to low concentrations of gaseous ethoxysilicates. This allows the removal of the hydrolysis product ethanol and prevents damage to the immobilized cells. [49] Campostrini et al. immobilized plant cells on modified glass fibers. [48] Sglavo et al. performed similar immobilizations upon collagen fibers. [20]

Despite the promise of sol-gel-derived materials, limited progress in the use of sol-gel-derived materials as a cell immobilization matrix has been made. Common sol-gel production methods are too cytotoxic at the time of gelation for extensive use in the immobilization of cells. Furthermore, macroporous samples amenable to colonization are difficult to obtain and may require the use of toxic chemicals. In the above-described references, sol-gel-derived immobilization matrices were either rinsed and subsequently loaded with a cytocompatible liquid phase after gelation but before loading with microorganisms [7] or they were limited to robust species that could survive the relatively harsh conditions at gelation. [8][9][10] Similar problems with the immobilization of other biological materials like biopolymers (e.g., proteins and nucleic acids) have arisen. Many biopolyrners readily denature under even the relatively tame conditions of many common sol-gel-derived material production routes. Furthermore, macroporous samples that provide facile mass transport often require the harsher production conditions, including increased concentrations of organic solvents and increased temperatures, [31][32][33][34] than common meso- and microporous production routes and hence compatibility with biological materials is impaired. Moreover, it is extremely difficult to make robust monolithic macroporous samples using the traditional approaches to the production of macroporous gels.

Organic solvents are a common feature of almost all sol-gel production routes (including those that have been used for cell immobilization), and their elimination can significantly influence the properties of the gel. These organic solvents often serve multiple purposes in the production of sol-gel silica, including decreasing the polarity of the reaction solution to enable solvation of the alkoxy silicate precursor,[21] acting as a volume “place holder” to enable the production of gels with sufficiently low density, [22] providing a source of reactant for the reverse silica solvation reaction during aging,[23] controlling drying during the production of optical-quality xerogels,[24][25] and acting as a polar phase in phase separation techniques. [26] The organic solvents are most commonly the alcohol corresponding to the alkoxy substituents on the silicate precursor, but other solvents have been chosen and often yield unique microstructural features.[22][24] However, high concentrations of organic solvents are often not compatible with biological materials and limit the utility of these production routes.

### SUMMARY OF THE INVENTION

Accordingly, one object of this invention is to provide a method and a composition of matter for the immobilization of biological materials, the method and composition being compatible with said biological materials.

Another object of this invention is to provide a method and a composition of matter for the formation of mechanically robust macroporous gels.

As used herein, a “biological material” refers to any cell, biopolymer (including, e.g., proteins, enzymes, nucleic acids, antibodies, and fragments thereof), cellular component, tissue, ligand, and/or combination thereof.

As used herein, immobilization refers to physical, chemical, and/or mechanical fixing to the sol-gel-derived matrix. In other words, immobilization includes, e.g., covalent bonding, ionic bonding, hydrogen bonding, Van der Waals interactions, hydrophobic interactions, and/or specific and non-specific recognition and binding, as well entrapment and entanglement that lead to at least some retention of biological material(s) by the sol-gel-derived matrix.

As used herein, “compatible with biological materials” connotes increased cytocompatibility, decreased denaturing of proteins, decreased incidence of lysing and/or cleaving of biological material(s), and/or reduced death of, damage to, destruction of, and/or disordering of biological material(s). Although there is a wide variability in robustness of biological materials, as well as the kind and degree of damage to biological materials, compatibility as used herein refers to an solvent that is more than 71 mole % water, more preferably more than 86 mole % water, even more preferably more than 86 mole % water, and most preferably more than 96% water.

These and other objects of the invention are provided by a method and a sol that can be used to form gels that are compatible with biological materials and/or robust and macroporous. As needed, the two step nature of the gelation reaction can be exploited to allow removal of undesired organic solvents such as hydrolysis reaction by-products from an acidic aqueous sol prior to gelation. Thus, sols that are substantially free of organic solvents and compatible with biological materials can be produced. Also as needed, robust, macroporous gels can be made by introducing water-soluble organic polymers to similar sols, with or without biological materials present.

A method according to the present invention thus includes hydrolyzing a sol-gel precursor in water to form a sol containing an organic solvent; removing said organic solvent from said hydrolyzed sol; and mixing said biological material with said hydrolyzed sol after said removing step.

Another method according to the present invention includes providing a sol substantially free from organic solvents to an extent sufficient to make said sol compatible with a biological material; and immobilizing said biological material by mixing said biological material into said sol.

Another method according to the present invention includes hydrolyzing a sol-gel precursor in water to form a sol containing an organic solvent; mixing said biological material with said sol; mixing a sufficient amount of a dispersant into said sol to cause macropores in a gel formed by said sol.

A sol according to the present invention thus includes a species formed by the hydrolysis of P moles of a sol-gel precursor; W moles of water; and a biological material, wherein said sol is substantially free from organic solvents to an extent sufficient to make said sol compatible with said biological material.

Another sol according to the present invention thus includes a species formed by the hydrolysis of P moles of a sol-gel precursor; W moles of water; a sufficient amount of a dispersant to cause macropores in a gel formed by said sol; and a biological material.

The present invention thus provides a method and a composition of matter that yield sols that are compatible with biological materials and/or are robust and macroporous. In order to produce sufficiently porous gels in the lack of an organic solvent “place holder,” the hydrolysis ratios (molar ratio of water to alkoxy silicate) investigated here are higher than in common sol-gel recipes. To the best of our knowledge, such high hydrolysis ratio sols have only been the subject of limited investigations, [21] and several researchers have failed to obtain quality silica gels at even lower hydrolysis ratios. The primary reason for this failure appears to be difficulty in obtaining sufficient solubility of the alkoxy silicate in relatively polar aqueous phases. This issue has been addressed by the current production scheme by hydrolyzing the alkoxy silicate in a low pH aqueous solution until it is sufficiently polar to completely dissolve in the aqueous solvent. Once dissolution has occurred, the hydrolyzed sol is amenable to further manipulation to improve compatibility with biological systems and manipulate microstructure for specific applications. The hydrolyzed sols described here were all distilled to remove as much organic solvent (e.g., hydrolysis reaction by-product alcohol) as possible prior to the addition of biological species. Other researchers have indicated that elevated temperatures during hydrolysis may provide the additional advantage of more precise control over the nature of the hydrolysis products. [27][22]

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 illustrates an exemplary process flow for performing the present invention;

FIG. 2 provides UV-vis transmittance spectra of various aerogels;

FIGS. 3A, 3B, and 3C respectively graphically illustrate the BET surface area, BJH adsorption cumulative pore volume, and BJH adsorption mean pore diameter as a function of PEG concentration in various aerogels;

FIG. 4 graphically illustrates the influence of PEG concentration upon nitrogen sorption isotherms of various aerogels;

FIG. 5 graphically illustrates the influence of “aging” the silica sol upon nitrogen sorption isotherms of various aerogels;

FIGS. 6A, 6B, and 6C respectively illustrate the influence of time of gelation after completion of distillation upon the BET surface area, BJH adsorption cumulative pore volume, and BJH adsorption mean pore diameter of various aerogels;

FIG. 7 graphically illustrates the influence of time of gelation after distillation upon apparent longitudinal acoustic velocity in various aerogels;

FIG. 8 graphically illustrates the influence of temperature at gelation upon nitrogen sorption isotherms of various aerogels;

FIGS. 9A, 9B, and 9C graphically respectively illustrate the influence of temperature at gelation upon BET surface area, BJH adsorption cumulative pore volume, and BJH adsorption mean pore diameter of various aerogels;

FIG. 10 graphically illustrates longitudinal acoustic velocity as a function of temperature at gelation of various aerogels;

FIG. 11 graphically illustrates the influence of PEG concentration upon nitrogen sorption isotherms of various aerogels;

FIG. 12 graphically illustrates the influence of hydrolysis ratio of exemplary sols upon the mean density and standard deviation of density of various as-dried aerogels; and

FIGS. 13A, 13B, and 13C graphically and respectively illustrate BET surface area, BJH adsorption cumulative pore volume, and BJH adsorption mean pore diameter as a function of PEG concentration in various aerogels.

### DETAILED DESCRIPTION OF THE PREFERRED EMBODIMENTS

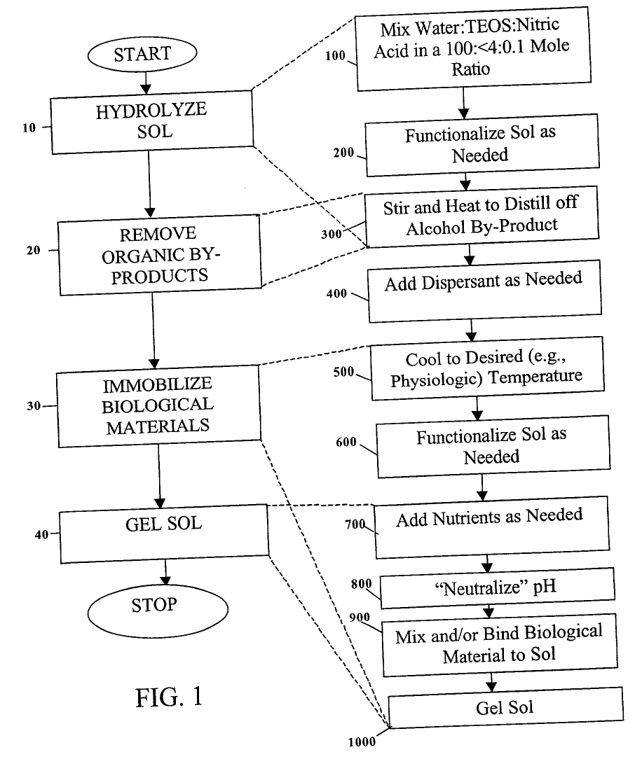

Referring now to the drawings, wherein like reference numerals designate identical or corresponding parts throughout the several views, and more specifically to FIG. 1 thereof, wherein an exemplary process flow for performing the present invention is illustrated. Generic descriptions of process flow steps are provided in the left hand column of FIG. 1 (steps 10, 20, 30, and 40), while exemplary specific process flow steps (steps 100, 200, 300, 400, 500, 600, 700, 800, and 900) are given in the right hand column of FIG. 1, along with dashed lines crossing between the two columns indicating corresponding items. Naturally, as described herein, the correspondence between the two columns, and the order of the steps within each column, can be changed within the scope of the present invention. For example, the mixing of relatively small amounts of water-soluble organic polymers, illustrated in step 400, can be performed prior to step 100 if needed, or it can be considered as corresponding to step 30 if the immobilized biological material is immobilized by entrapment and/or entanglement within the final gel. Another example of the potential multiple order and purpose behind the illustrated steps is expressly indicated by the repetition of “Functionalize Sol as Needed” in steps 600 and 200.

In step 10, the sol is hydrolyzed. Hydrolysis occurs spontaneously upon the exposure of several common sol-gel precursors to water, and commonly involves the formation of a hydroxy metallate. Furthermore, several hydrolysis reactions yield organic solvent by-products, such as ethanol and/or acetic acid, although this is not necessarily the case. For example, the hydrolysis byproduct for chlorosilanes is hydrochloric acid.

If the hydrolysis reactions yields an organic solvent that is not compatible with the biomaterial of interest, it is desirable that the hydrolysis reaction be driven to completion. Hydrolysis can be driven, e.g., by increased temperatures, amounts of water, and/or exposure to acidic pH's. Strong acids are commonly used to catalyze the hydrolysis reaction in aqueous solutions. In some embodiments, the pH of the hydrolysis solution is below 4. More preferably, the pH of the hydrolysis solution is below 3. Moreover, in addition to providing sol-gels with desirable transport and mechanical properties, distillation of high-hydrolysis sols also acts to ensure more complete hydrolysis of the sol-gel precursor.

The present invention is safely and relatively inexpensively implemented using tetraethylorthosilicate (TEOS, Aldrich Chem) as the sol-gel precursor. TEOS hydrolyzes to various mildly acidic silicic acid species. Other sol-gel precursors can readily be used in accordance with the present invention. For example, other alkoxysilicates such as tetramethylorthosilicate (TMOS, Aldrich Chem.), alkoxytitanates, and/or alkoxyaluminates [42] can be substituted for TEOS in step 100.

Sols with hydrolysis ratios (moles water per mole sol-gel precursor) above 25 (e.g., 4 or fewer moles TEOS per 100 moles water) were found to reproducibly yield mechanically stable wet gels with adequate transport properties. Naturally, sols with other hydrolysis ratios can be used, depending upon the desired mechanical robustness, transport properties, and pore size of the desired gels. In general, as hydrolysis ratio increases, the time needed for gelation increases, but density and resistance to mass transport of product gels decreases. For these reasons, sols with hydrolysis ratios above 50 are more desirable. Sols with hydrolysis ratios above 100 yield gels that are suitable for low mechanical load applications, and sols with hydrolysis ratios above 200 are even more preferable for low mechanical load applications. In our lab, gels with hydrolysis ratios greater than 25 were amenable to reproducible pore size manipulation using polyethylene glycol, a water-soluble polymer that has been tested by others in various biological applications and possesses proven biocompatibility with many biological systems. Moreover, the microstructures of the final gels were reproducibly tunable and the production of monolithic macroporous gels and aerogels was possible. Naturally, other water-soluble polymers and other dispersants are capable of providing similar pore size manipulation.

If desired, the hydrolyzed sols can be functionalized as needed (step 200) during hydrolysis. For example, any of a variety of silane coupling agents [43][44] such as aminopropyltriethoxysilane (APTES) and propyltrichlorosilane (PTS) can be added to the aqueous solution for functionalizing the sol and/or the gel before, during, or after hydrolysis. Some adjustment of process parameters may be necessary, depending upon the nature of the added silane coupling agent. For example, if the sol is to be functionalized with APTES, then an additional (e.g., molar equivalent) amount of acid can be added to the sol to compensate for the basicity of APTES. Likewise, if the sol is functionalized with PTS, then less nitric acid or even additional base (e.g., 3 moles less acid/more base per mole PTS) can be added to the sol to compensate for the acid generated by the hydrolysis of PTS. Depending upon the amount and the degree of functionalization, the porous structure of aerogels produced from a functionalized sol may also change.

Functionalization (step 200) can be performed for any of a number of reasons, including to change the phase behavior of the hydrolyzed sol and to prepare the sol for covalent attachment to the biological material(s). For example, a sol that has been functionalized with APTES can form covalent (Schiff base) bonds with biological materials that contain aldehyde groups. A sol that has been functionalized with a mercapto-(i.e., thiol-) containing moiety can form disulfide bridges to biological materials that also contain mercapto (thiol) functionalities.

The distillation illustrated in step 300 performs the dual purpose of facilitating hydrolysis of the sol and removing the organic by-products of the hydrolysis reaction, and hence corresponds to both steps 10 and 20. However, this is not necessarily the case, since, for example, the sol-gel precursor could be added dropwise to the water/nitric acid mixture and hydrolysis of the sol-gel precursor (step 10) substantially completed prior to removal of organic by-products (step 20). Also in accordance with the present invention, the distillation conditions relating to step 300 for the removal of the ethanolic by-product of the hydrolysis reaction can be changed as needed. For example, a reflux distillation can be performed as needed.

Either before or after the solution is allowed to return to the desired temperature (step 500), a dispersant can be added (step 400). Exemplary dispersants include water soluble organic polymers and organic species that increase the viscosity of the sol. An exemplary water-soluble polymer is polyethylene glycol in amounts between about 0 and 5 grams per 100 ml solution, and more preferably between 0 and 2.5 grams per 100 ml solution. Other exemplary dispersants include polyethylene oxide and glycerol. The dispersant can be dissolved through vigorous shaking if needed.

Cooling the sol to a desired temperature (step 500) is performed primarily to maximize compatibility between the sol and the biological material to be immobilized, and hence corresponds to the immobilization of the biological material (step 30). For example, many proteins denature at elevated temperatures, and many bacteria die at elevated temperature. Naturally, the desired temperature can be selected based upon the requirements of the biological material of interest.

In step 500, the sol can be functionalized as needed in a manner similar to that described in regard to step 600. Functionalization can readily be accomplished using any of a variety of silane coupling agents for any of a variety of different purposes. If the functionalization involves the binding of temperature sensitive moieties, then functionalization should be performed in step 600 rather than step 200.

In step 700, cellular nutrients can be added to the sol if needed, e.g., if cellular and/or tissue biological materials are to be immobilized within the cell. For example, a cytocompatible amount of (buffered or unbuffered) growth medium powder can be added to the hydrolyzed sol and dissolved by rapid shaking. If the cellular nutrients have a sufficient buffering capacity to raise the pH of the sol into a range that is compatible with the biological materials of interest, then no further “neutralization” (as illustrated in step 800) need be performed. The biological materials can then be directly added (step 900), and gelation (step 1000) can, in certain systems occur. In the case of entrapment and/or entanglement of certain biological materials, gelation physically immobilizes the biological materials and hence step 700 is indicated as potentially corresponding to both the immobilization of biological materials (step 30) and the gelation of the sol (step 40). Likewise, if the buffering capacity and nutrient content of the carrier solution that contains biological materials is sufficient to raise the pH of the sol into a range that is compatible with the biological materials of interest and to supply the biological materials with necessary nutrients, then both step 700 and the neutralization of step 800 can be dispensed with.

In step 800, the pH of the sol is “neutralized.” As used herein, “neutralization” refers to changing the pH of the sol to a range compatible with the biological material of interest, rather than indicating that the pH of the sol is changed to 7. As such, “neutralization” often indicates bringing the pH of the sol into physiological ranges. For example, “neutralization” often involves compensating for the acid used to promote hydrolysis in step 100 by the addition of a molar equivalent amount of base. Since the hydrolyzed sol-gel precursors themselves are often mildly acidic (for example, silicic acid species), the pH of the sol is not raised to 7 by this procedure. Rather, the pH is simply brought into a range that is tolerable to many microorganisms and other types of biological matter. As another example, if the biological material is acidophillic and the pH of the sol is already in a range that is compatible with the acidophillic biological materials, then “neutralization” has already occurred and no further modifications are needed.

In most sols, if “neutralization” raises the pH of the sol above 5 or so, gelation will occur almost immediately. Since the condensation reaction that forms the gel matrix is much more rapid under neutral and/or basic rather than acidic conditions, “neutralizing” the pH often serves the dual purpose of preparing the sol for reception of the biological material and initiating gelation. This is very convenient, since many acidic hydrolyzed sols can be stored for long periods without gelation occurring. Thus, when immobilization of the desired biological material and gelation is desired, an aliquot can simply be drawn from an acidic hydrolyzed sol reservoir and treated as described in steps 700, 800, 900, and/or 1000 as needed.

After “neutralization” but prior to gelation, in step 900, the biological material(s) can be mixed and/or bound to the sol. The mixing and/or binding or the biological materials described in step 900 can occur by any of a number of different mechanisms. For example, as Pope pointed out, the mean pore diameter of traditional sol-gel-derived silica gels is two orders of magnitude smaller than the dimensions of common cells.[12] In the absence of macropores, any cell present in the sol at the time of gelation is thus immobilized by entrapment within the gel. [19] Naturally, adhesion promoters such as traditional extracellular matrix components such as Type I collagen, Type IV collagen, laminin, Matrigel, various proteoglycans such as heparin sulfate and dermatan sulfate, fibronectin, and combinations thereof can be covalently bound to the sol during, e.g., steps 200 and/or 600, and used to affix a cellular and/or tissue biological material to the product gel even when macropores are present in the gel. Other potential adhesion promoters include engineered components such as Pronectin-F, a recombinant protein containing multiple copies of the RGD cell attachment ligand of human fibronectin interspersed between repeated structural peptide segments derived from spider silk, thereby providing a non-degradable, highly stable substrate for cell attachment.

Biological materials such as biopolymers can also be entrapped and/or bound to the gel matrix, [45][46][47] as well as covalently bound using both regiospecific and non-regiospecific immobilization procedures. Many such procedures are well known in the art, and commonly involve functionalization of the sol with a silane coupling agent at some point during processing.

In step 1000, the sol can be gelled. As indicated above, any one of steps 700, 800, or 900 can spontaneously lead to gelation under selected conditions. However, this is not necessarily the case, and other steps, such as evaporating a portion of the sol and/or waiting for relatively long periods of time can also be used to gel the sols. Since acidic hydrolyzed sols will, with time, spontaneously gel, the pH of the sols need not necessarily be raised in order to perform step 1000.

Several exemplary embodiments of the above-described method and sols will now be described, as will the characteristics of gels formed using these methods and sols.

Embodiment #1:

200 ml of distilled water were heated to approximately 60 degrees C. while adding 0.4 ml of 70% nitric acid and 75 ml of tetraethylorthosilicate (TEOS) and stirring vigorously. This was continued past the point where only a single phase was visible (typically after approximately 3 hrs.). The sol was then cooled to physiological temperature range. To 15 ml of this solution, 0.4 g of Carbowax Sentry polyethylene glycol 3350 flake was added, and the sol was stirred vigorously. 0.5 g of LB Broth was then added and stirred vigorously, and 5 ml of cell solution in comparable broth were then immediately added while stirring. A thick film of the sol was formed by pipetting drops on an activated glass microscope slide, and the sol was then gelled. After gelation, the slide was placed in nutrient solution to provide nourishment reservoir for cells. If monoliths are formed, they can be cooled to lower cell metabolic rate and extend shelf-life of cells in the matrix if needed.

Embodiment #2

200 ml of distilled water were heated to approximately 60 degrees C. while adding 0.4 ml of 70% nitric acid and 75 ml of tetraethylorthosilicate (TEOS) and stirring vigorously. Heating continued past the point where only a single phase (typically after approximately 3 hrs.) was visible, and then the sol was cooled to the physiological temperature range. To 15 ml of this solution, 0.15 g of Carbowax Sentry polyethylene glycol 3350 flake was added. This was then stirred vigorously. 0.5 g of LB Broth was then added and stirred vigorously. Next, 5 ml of cell solution in comparable broth was immediately added while stirring. A thick film was formed by pipetting drops on an activated glass microscope slide. After gelation, the slide can be placed in a nutrient solution to provide a nourishment reservoir for cells. If monoliths are formed, they can be cooled to lower the metabolic rate and extend the shelf-life of cells in the matrix if needed.

Embodiment #3:

200 ml of distilled water was heated to approximately 85 degrees C. while adding 0.4 ml of 70% nitric acid and 75 ml of tetraethylorthosilicate (TEOS) and stirring vigorously. Distillation continued for approximately 1 hr. The solution was the cooled to the physiological temperature range, and 20 ml was added to 0.9 ml of 1M potassium hydroxide. This was then stirred vigorously, and immediately 5 ml of cell solution in comparable broth was added while stirring. A thick film can be formed by pipetting drops onto an activated glass microscope slide. After gelation, the slide can be placed in nutrient solution to provide a nourishment reservoir for cells. If monoliths are formed, they can be cooled to lower the metabolic rate and extend the shelf-life of the cells in the matrix if needed.

Embodiment #4:

200 ml of distilled water can be heated to approximately 85 degrees C. while adding 0.4 ml of 70% nitric acid and 75 ml of tetraethylorthosilicate (TEOS) and stirring vigorously. This can be distill for approximately 1 hr, and then cooled to the physiological temperature range. To 20 ml of this solution, 0.2 g of Carbowax Sentry polyethylene glycol 3350 flake can be added while stirring vigorously. Next, 0.9 ml of 1 M potassium hydroxide can be added while stirring vigorously, and then 5 ml of cell solution in comparable broth can be immediately added while stirring. A thick film can be formed by pipetting drops on an activated glass microscope slide. After gelation, the slide can be placed in nutrient solution to provide a nourishment reservoir for cells. If monoliths are formed, they can be cooled to lower the metabolic rate and extend the shelf-life of the cells in the matrix if needed.

Further Embodiments:

High hydrolysis ratio hydrolyzed sols were prepared by heating a 100:0.1 molar ratio of distilled water:nitric acid solution to the distillation temperature (either 60 degrees C. or 85 degrees C.), followed by adding a sufficient volume of chilled (4 degrees C.) tetraethylorthosilicate (TEOS, Aldrich Chem.) to obtain hydrolysis ratios of 25, 33, or 50. This mixture was initially turbid and stirred at 800 RPM for 10 minutes. This was usually sufficient to yield a transparent sol that was stirred at 400 RPM for the remainder of the distillation. An open pot distillation was performed for 1 hr at 85 degrees C. or 18 hrs at 60 degrees C. The solution was allowed to return to room temperature, at which time polyethylene glycol (Carbowax Sentry Polyethylene Glycol 3350, DuPont) was added in amounts between 0 and 2.5 grams per 100 ml solution. The polyethylene glycol (PEG) was dissolved through vigorous shaking.

Gelation was induced by raising the pH of the acidic sol to form so-called “two-step” gels at room temperature immediately after solvation of the polymer. Furthermore, the influence of time and temperature at gelation was investigated. Temperature effects were investigated by heating or cooling aliquots of the same hydrolyzed sol/PEG mix to the desired temperature before proceeding with gelation. Time effects were investigated by allowing the distilled, hydrolyzed sol to “age” by stirring at room temperature in the absence of PEG. When the desired time had been reached, PEG was added to an aliquot of the sol before proceeding with gelation.

For the majority of the embodiments described herein, the experimental focus was the reproducible handling of these sols and characterization of the resultant gels rather than handling biological systems. Molar equivalent amounts (to nitric acid) of 1M aqueous potassium hydroxide solution were added to the polymer-containing, hydrolyzed sols. This induced gelation prior to the addition of either cells or growth media to the sol. This was done because growth media and cell solution composition is largely unknown and may vary or contain other constituents such as dispersants that influence the final microstructure of the gel. Gel times were commonly on the order of one minute and decreased with increasing polymer concentration. All gels were aged in their production solution for four days at room temperature and displayed significant syneresis. The range of stoichiometries and production conditions examined in this study is given in Table 1.

Many of the available gel characterization techniques (e.g., nitrogen sorption, SEM) require dry samples. In order to preserve as much of the microstructure of the wet gels as possible, the gels were supercritically dried to form aerogels. Aging in production solution was followed by three successive ethanol exchanges over a total of six days, and then yet another exchange with liquid carbon dioxide followed by supercritical drying to obtain the final aerogel samples.

Immediately after supercritical drying, the gels were weighed and the geometric dimensions measured manually. After an 18 hr evacuated bake at 250 degrees C., the mass of the gels was again measured and these values were assumed to correspond to an approximate density before and after removal of residual volatile contaminants from the gels. Aerogel samples were characterized through nitrogen sorption porosimetry, scanning electron microscopy (SEM), UV-vis spectroscopy, and acoustic velocity measurements. Nitrogen sorption porosimetry was performed using a Micrometrics ASAP Pore Size Analyzer on approximately 0.1 gram aerogel samples after an 18 hr evacuated bake at 250 diameter were calculated from the adsorption isotherms. SEM micrographs were taken using a JEOL JSM-35 after vacuum sputtering with approximately 200 angstroms of gold. UV-vis spectroscopy was performed on samples cast and aged in standard 10 mm/3ml polystyrene cuvettes. After aging, the samples were decast from the cuvettes, subject to ethanol exchange, supercritically dried, and then placed into Suprasil 300 quartz cuvettes and probed using a Hewlett-Packard

TABLE 1

| | | | Temperature | Sol “Age” | Distillation |
|---|---|---|---|---|---|
| Gel | Hydrolysis | PEG | at Gelation | at Gelation | Temp/Time |
| Num. | Ratio | (w/v %) | (° C.) | (hrs.) | (° C./hrs.) |
| 1 | 33 | 0 | 22 | 0 | 85/1 |
| 2 | 33 | 0.11 | 22 | 0 | 85/1 |
| 3 | 33 | 0.30 | 22 | 0 | 85/1 |
| 4 | 33 | 0.49 | 22 | 0 | 85/1 |
| 5 | 33 | 1.05 | 22 | 0 | 85/1 |
| 6 | 33 | 1.50 | 22 | 0 | 85/1 |
| 7 | 33 | 0.26 | 22 | 0 | 85/1 |
| 8 | 33 | 0.705 | 22 | 0 | 85/1 |
| 9 | 33 | 1.045 | 22 | 0 | 85/1 |
| 10 | 33 | 1.58 | 22 | 0 | 85/1 |
| 11 | 33 | 0.26 | 22 | 18 | 85/1 |
| 12 | 33 | 0.705 | 22 | 18 | 85/1 |
| 13 | 33 | 1.045 | 22 | 18 | 85/1 |
| 14 | 33 | 1.58 | 22 | 18 | 85/1 |
| 15 | 33 | 0.705 | 22 | 25 | 85/1 |
| 16 | 33 | 1.045 | 22 | 25 | 85/1 |
| 17 | 33 | 0.5 | 8 | 0 | 85/1 |
| 18 | 33 | 0.5 | 16 | 0 | 85/1 |
| 19 | 33 | 0.5 | 22 | 0 | 85/1 |
| 20 | 33 | 0.5 | 34 | 0 | 85/1 |
| 21 | 33 | 0.5 | 43 | 0 | 85/1 |
| 22 | 33 | 0.2 | 22 | 0 | 60/18 |
| 23 | 33 | 0.65 | 22 | 0 | 60/18 |
| 24 | 33 | 1.25 | 22 | 0 | 60/18 |
| 25 | 33 | 2.15 | 22 | 0 | 60/18 |
| 26 | 50 | 0.15 | 22 | 0 | 85/1 |
| 27 | 50 | 0.35 | 22 | 0 | 85/1 |
| 28 | 50 | 0.60 | 22 | 0 | 85/1 |
| 29 | 50 | 0.80 | 22 | 0 | 85/1 |
| 30 | 25 | 0.375 | 22 | 0 | 85/1 |
| 31 | 25 | 0.85 | 22 | 0 | 85/1 |

H.-P. 8453 UV-vis Spectrophotometer. Due to shrinkage during aging and/or supercritical drying, the final pathlength through the aerogel samples was 0.9011±0.008 cm Pulse transit time measurements of acoustic velocity were made on samples under ambient conditions using a Panametrics Pulser/Receiver 5055PR and ultrasonic preamplifier in conjunction with a matched pair of 5 MHz contact mode transducers (Panametrics, Model # V109). Coupling with the gel samples was performed using a single Parafilm layer placed between the transducers and the gels. The strain variability that has previously been observed in gel samples [35][36] was accommodated by measuring at the lowest possible stress that yielded an apparent signal.

Gelation can also be induced by addition of a cytocompatible amount of (buffered) growth medium powder to the hydrolyzed sol, followed by addition of the biological system of interest. In further embodiments, Luria-Bertani broth powder (Difco), containing yeast extract (5g/L), NaCl (10 g/L) and tryptone (10 g/L), was added at the manufacturers recommended concentration of 2.5 gl 100 ml to the hydrolyzed sol. The powder was dissolved by rapid shaking, 0.6 ml of 1 M KOH was added to bring the pH of the sol up to 6.0, and 0.5 ml of E. coli bacteria per 20 ml sol was immediately added. The bacteria had been grown to mid-logarithmic phase, which corresponded to a concentration of 10¹³ cells/ml. Viability of the bacteria within the resulting gel was determined microscopically using LIVE/DEAD Baclight bacteria viability kit (Molecular Probes, Eugene, Oreg.) following the manufacturer's instructions.

Characterization of the gels was performed using a number of different analytical techniques. SEM microscopy provided clear images of macrostructural features, whereas nitrogen sorption was used to probe the mesopore regime. UV-vis transmittance measurements probed features in both the macro- and mesopore regime. Acoustic velocity measurements provided a very sensitive probe of gel features, although correlating velocity measurements with discrete microstructural features proved difficult.

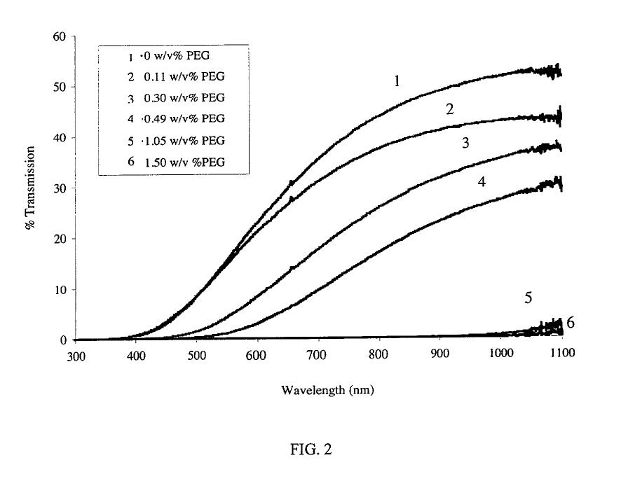

FIG. 2 graphically illustrates the influence of PEG concentration upon UV-vis transmittance spectra of various aerogels formed from gels using exemplary sols and exemplary methods for forming a sol. The gels were cast into optical cuvettes immediately after a one hour distillation at 85 degrees C. and had a hydrolysis ratio of 33. The decrease in transmissivity with increasing PEG concentration is presumably due to increased feature size and scattering.

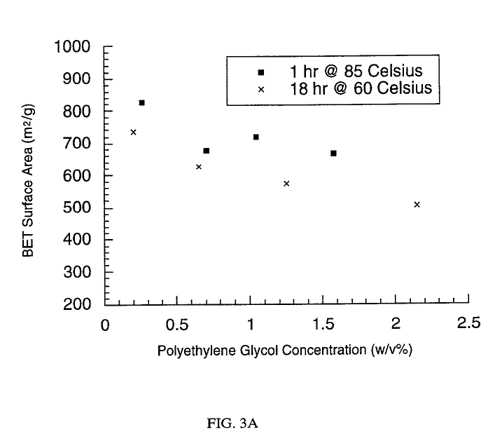

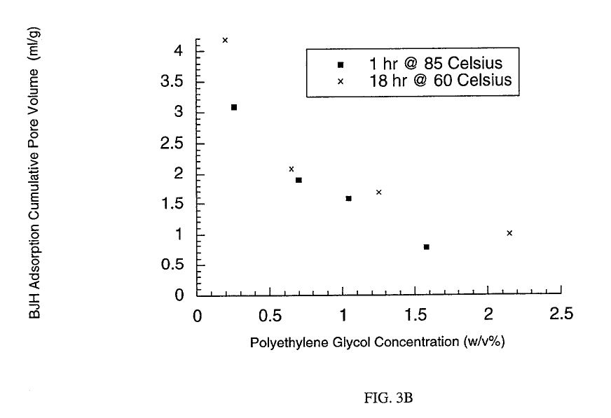

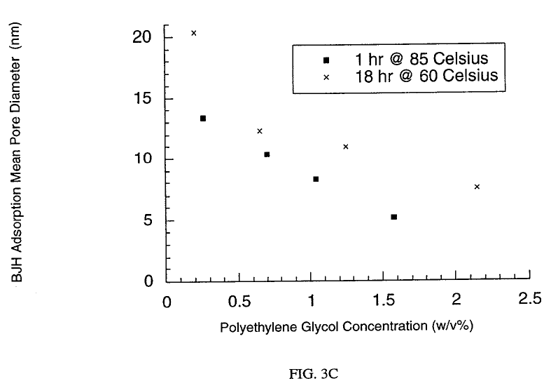

FIGS. 3A, 3B, and 3C. respectively graphically illustrate the BET surface area, BJH adsorption cumulative pore volume, and BJH adsorption mean pore diameter as a function of PEG concentration of various aerogels formed from gels using exemplary sols and exemplary methods for forming a sol. The BET surface area, BJH adsorption cumulative pore volume, and BJH adsorption mean pore diameter were determined from nitrogen sorption experiments. The measured values of all three decrease with increasing PEG concentration, regardless of distillation conditions. Measured pore volume (FIG. 3B) and mean pore diameter (FIG. 3C) decrease with increasing PEG concentration. However, since the density of the gels is nearly identical, this probably represents an increased fraction of the porosity shifting outside the mesopore range. A longer and lower temperature distillation appears to produces gels with an increased fraction of the porosity still within the mesopore range.

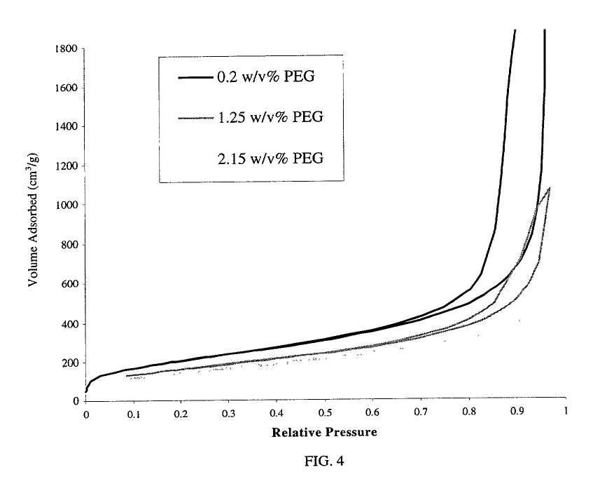

FIG. 4 graphically illustrates the influence of PEG concentration upon nitrogen sorption isotherms of various aerogels formed from gels using exemplary sols and exemplary methods for forming a sol. The aerogels had a hydrolysis ratio of 33 and were gelled after an 18 hour distillation at 60° C. As polyethylene glycol concentration increases, the amount of nitrogen sorption decreases. It is believed that this is a consequence of an increased fraction of the porosity shifting outside the mesopore range.

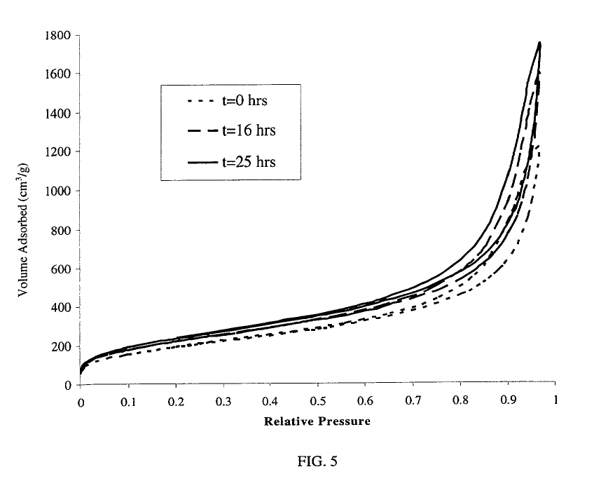

FIG. 5 graphically illustrates the influence of “aging” the silica sol upon nitrogen sorption isotherms of various aerogels formed from gels using exemplary sols and exemplary methods for forming a sol. The sols had a hydrolysis ratio of 33, a PEG concentration of 0.705 w/v %, and were distilled for one hour at 85° C. and cooled to room temperature before time was set to zero. “Neutralization” of the acidic hydrolysis catalyst was performed at the times indicated and gelation proceeded immediately. In this particular grouping, as time progressed, the amount of nitrogen adsorption increases. It is believed that this is a consequence of increased polycondensation of the hydrolyzed sol with time.

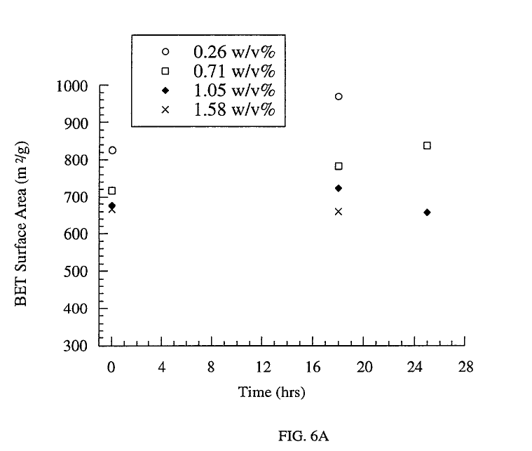

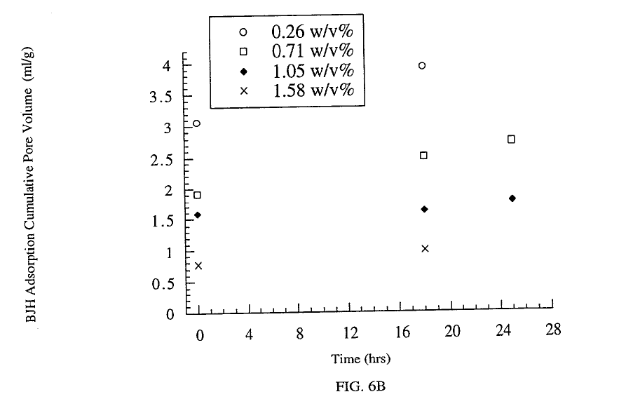

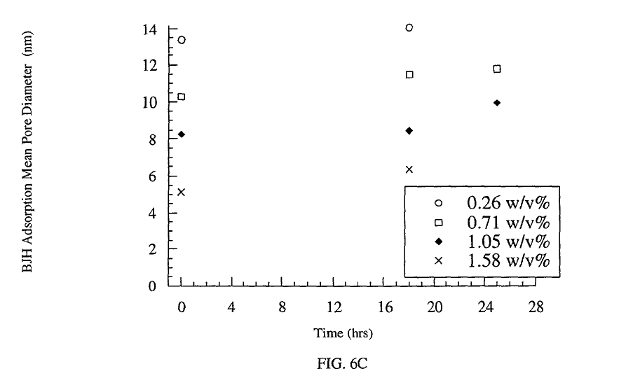

FIGS. 6A, 6B, and 6C. respectively illustrate the influence of time of gelation after completion of distillation upon the BET surface area, BJH adsorption cumulative pore volume, and BJH adsorption mean pore diameter of various aerogels formed from gels using exemplary sols and exemplary methods for forming a sol. The influence of distillation time upon the apparent surface area (FIG. 6A) of the aerogels is difficult to identify over these times. The apparent pore volume (FIG. 6B) and mean pore diameter (FIG. 6C) of the aerogels appears to increase slightly with increasing sol “aging” time, regardless of PEG concentration.

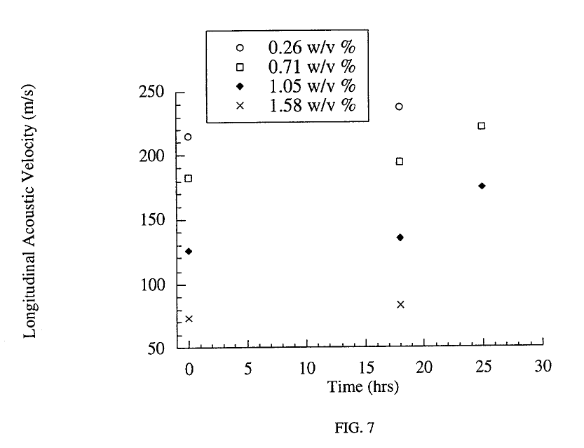

FIG. 7 graphically illustrates the influence of time of gelation after distillation upon apparent longitudinal acoustic velocity in various aerogels formed from gels using exemplary sols and exemplary methods for forming a sol. Interestingly, acoustic velocity measurements provide the clearest indication of the influence of sol “aging” time. The increased acoustic velocity may be related to increased polycondensation of the hydrolyzed sol under acidic conditions, as gels formed under such conditions tend to possess higher elastic moduli.

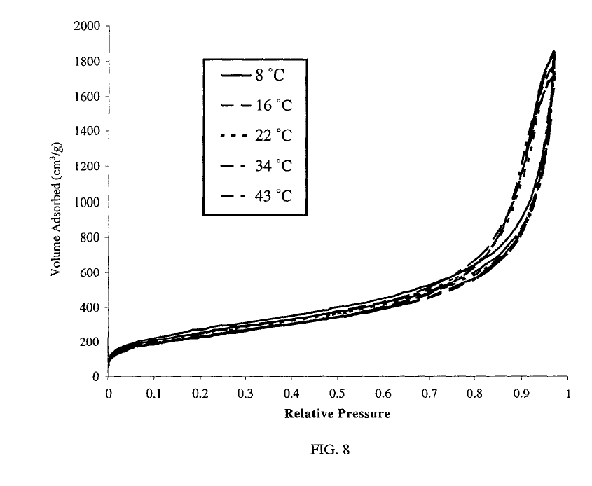

FIG. 8 graphically illustrates the influence of temperature at gelation upon nitrogen sorption isotherms of various aerogels formed from gels using exemplary sols and exemplary methods for forming a sol. These exemplary sols had a hydrolysis ratio of 33, a PEG concentration of 0.5 w/v %, and were distilled for one hour at 85° C. “Neutralization” of the acidic hydrolysis catalyst was performed at the temperatures indicated. Although the differences between the gels were macroscopically apparent, the adsorption isotherms are nearly indistinguishable from one another.

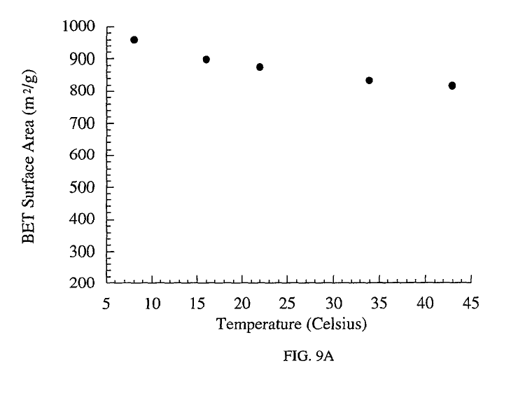

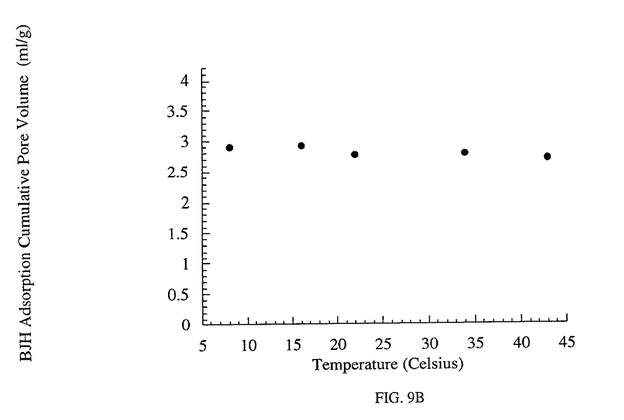

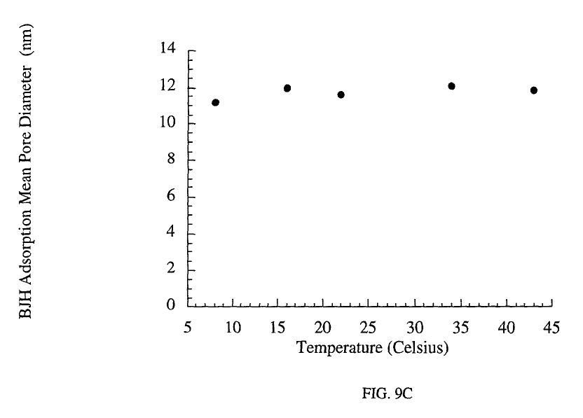

FIGS. 9A, 9B, and 9C. graphically respectively illustrate the influence of temperature at gelation upon BET surface area, BJH adsorption cumulative pore volume, and BJH adsorption mean pore diameter of various aerogels formed from gels using exemplary sols and exemplary methods for forming a sol. The BET surface area, BJH adsorption cumulative pore volume, and BJH adsorption mean pore diameter were calculated based upon nitrogen sorption measurements. The apparent surface area (FIG. 9A) and pore volume (FIG. 9B) of the aerogels may decrease slightly with increasing temperature, whereas mean pore diameter appears to be independent of temperature. These effects appear minimal relative to the influence of PEG concentration and are indicative of the robustness of the production route.

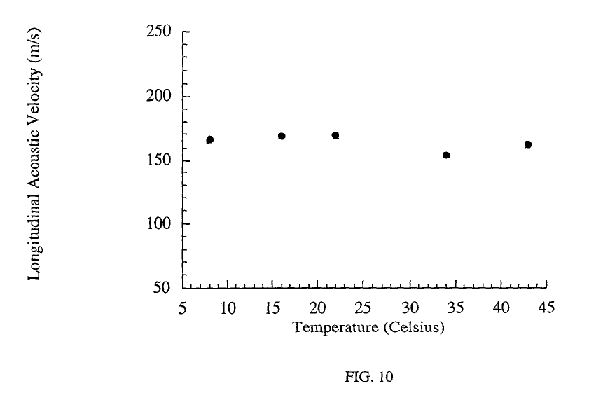

FIG. 10 graphically illustrates longitudinal acoustic velocity as a function of temperature at gelation of various aerogels formed from gels using exemplary sols and exemplary methods for forming a sol. The acoustic velocity also appears to be independent of temperature. The standard deviation of the velocity measurements is smaller than the marker size.

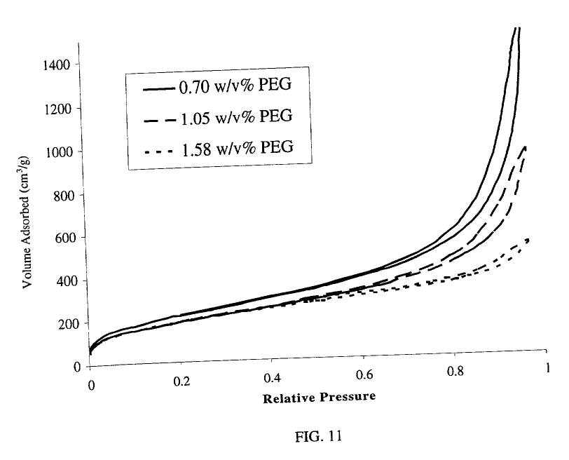

FIG. 11 graphically illustrates the influence of PEG concentration upon nitrogen sorption isotherms of various aerogels formed from gels using exemplary sols and exemplary methods for forming a sol. These sols had a hydrolysis ratio of 33 and were gelled after a one hour distillation at 85° C. As polyethylene glycol concentration increases, the amount of nitrogen sorption decreases. It is believed that this is a consequence of an increased fraction of the porosity shifting outside the mesopore range.

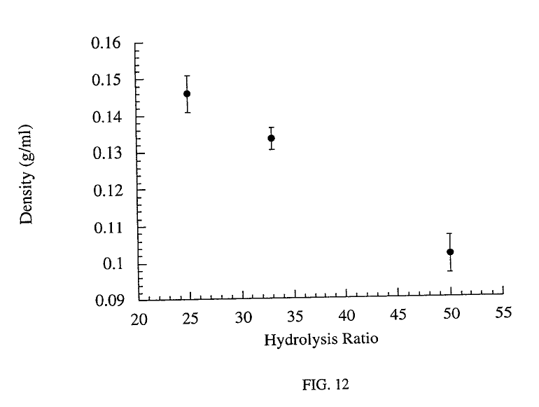

FIG. 12 graphically illustrates the influence of hydrolysis ratio of exemplary sols upon the mean density and standard deviation of density of various as-dried aerogels formed from gels using exemplary sols and exemplary methods for forming a sol. As hydrolysis ratio increases, density decreases. With water substantially the only volume “place holder” (non-silica constituent) in the sols, the decrease in density is due the lower volume fraction of silica in the sol.

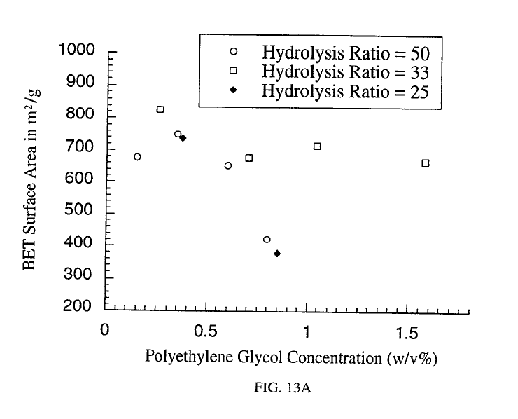

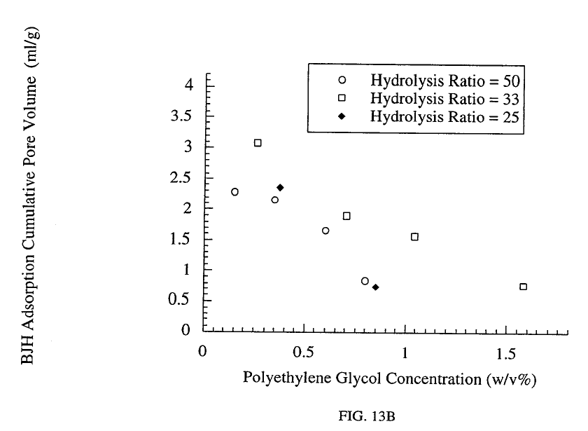

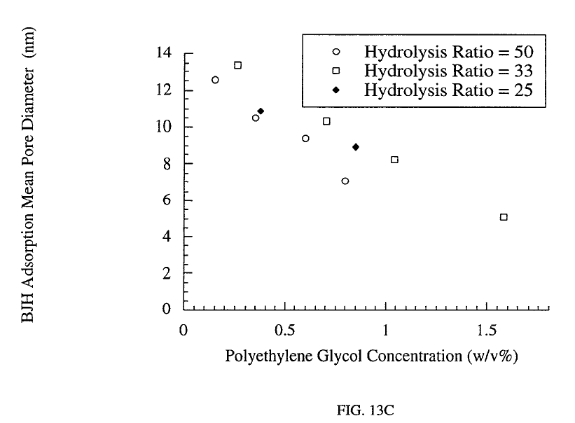

FIGS. 13A, 13B, and 13C. graphically and respectively illustrate BET surface area, BJH adsorption cumulative pore volume, and BJH adsorption mean pore diameter as a function of PEG concentration in various aerogels formed from gels using exemplary sols and exemplary methods for forming a sol. It appears that the surface area of the aerogels formed from sols with the lowest (25) and highest (50) examined hydrolysis ratios are more similar to each other than they are to the intermediate hydrolysis ratio (33) sols.

Cell viability can be determined using the BacLight live/dead staining kit and imaging using a fluorescent light microscope at 200× magnification of E. coli 24 hr after immobilization in a 0.5% PEG gel produced from an exemplary sol and an exemplary method for forming a sol. Live cells are labeled with green fluorescence, and dead cells are labeled with red fluorescence. Such micrographs indicate that the vast majority of the bacteria remain viable.

In summary, sol-gel materials have long held promise as immobilization matrices. By working in an aqueous hydrolyzed sol that contains essentially only hydrolyzed precursors to silica, compatibility of the sol-gel process with biological materials is dramatically improved. Furthermore, dispersants such as water-soluble polyethylene glycol can be added to hydrolyzed sols to produce robust macroporous gels. Indeed, as needed, both of these features can be combined to yield a sol-gel process that is both compatible with biological materials and yield robust, monolithic gels that may be amenable to colonization and provide facile mass transport.

### LIST OF REFERENCES

[1] S. I. Anderson, S. Downes, C. C. Perry, and A. M. Caballero, J. Mat. Sci. : Mat. Med. 9, 731 (1998).

[2] K. Tsuri, C. Ohtsuki A. Osaka, T. Iwamoto, and J. D. Mackenzie, J. Mat. Sci. : Mat. Med. 8, 157 (1997).

[3] S. -B. Cho, K. Nakanishi, T. Kokubo, N. Soga, C. Ohtsuki, and T. Nakamura, J. Biomed. Mat. Res. 33,141(1996).

[4] S. -B. Cho, F. Miyaji, T. Kokubo, K. Nakanishi, N. Soga, and T. Nakamura, J. Biomed. Mat. Res. 32, 375 (1996).

[5] V. Gross, R. Kline, H. J. Schmit7, and V. Strunz, CRC Crit. Rev. Biocomp. 4, 2 (1988).

[6] L. L. Hench in Bioceramics: Materials Characteristics versus In Vivo Behavior; Ducheyne, P., Lemmons, J., Eds.; .; (Ann. New York Acad. Sci., 1988) Vol. 523, p 54.

[7] M. Uo, K. Yamashita, M. Suzuki, E. Tamiya, I. Karube, and A. Makishima, J. Ceram. Soc. Jpn. 100, 426 (1992).

[8] G. Carturan, R. Campostrini, S. Dire, A. Scardi, and E. De Alterius, J. Mol. Catal. 57, L13 (1989).

[9] E. J. A. Pope, J. Sol-Gel Sci. Tech. 4, 225 (1995).

[10] M. Al-Saraj, M. S. Abdel-Latif, I. El-Nahal, and R. Baraka, J. Non- Cryst. Sol. 248, 137 (1999).

[11] T. Hino, H. Yamada, and S. Okamura, U.S. Pat. No. 4,148,689, 1979.

[12] E. J. A. Pope, K. Braun, M. Van Hirtum, C. M. Peterson, P. Tresco, and J. D. Andrade in Sol-Gel Science and Technology; Pope, E. J. A., Saika, S., Klein, L., Eds.; (Am. Ceramic Soc., Westerville, Ohio, 1995) Vol. Ceramic Transactions, Vol. 55, p 33.

[13] E. J. A. Pope, U.S. Pat. No. 5,693,513, 1997.

[14] E. J. A. Pope, U.S. Pat. No. 5,739,020, 1998.

[15] E. J. A. Pope, K. Braun, and C. M. Peterson, J. Sol-Gel Sci. Tech. 8, 635 (1997).

[16] K. P. Peterson, C. M. Peterson, and E. J. A. Pope, Proc. Soc. Exp. Bio. Med. 218, 365 (1998).

[17] E. J. A. Pope, U.S. Pat. No. 5,895,757, 1997.

[18] J. Livage, J. Y. Barreau, J. M. Da Costa, and I. Desportes, SPIE Proc. Ser. (Sol-Gel Optics III) 2288, 493 (1994).

[19] T. Branyik, G. Kuncova, J. Paca, and K. Demnerova, J. Sol-Gel Sci. Tech. 13, 283 (1998).

[20] V. M. Sglavo, G. Carturan, R. Dal Monte, and M. Muraca, J. Mat. Sci 34, 3587 (1999).

[21] L. C. Klein, and G. J. Garvey in Better Ceramics Through Chemistry: MRS Symp. Proc.; Brinker, C. J., Clark, D. E., Ulrich, D. R., Eds.; (North-Holland, N.Y., 1984) Vol. Vol. 32, p 33.

[22] T. M. Tillotson, J. F. Poco, L. W. Hrubesch, and I. M. Thomas, U.S. Pat. No. 5,409,683, 1995.

[23] J. K. West, R. Nikles, and G. Latorre in Better Ceramics Through Chemistry. MRS Symp. Proc.; Brinker, C. J., Clark, D. E., Ulrich, D. R., Eds.; (North-Holland, New York, 1988) Vol. 121, p 219.

[24] L. L. Hench, G. Orcel, and J. L. Nogues in Better Ceramics Through Chemistry: MRS Symp. Proc.; Brinker, C. J., Clark, D. E., Ulrich, D. R., Eds.; (North-Holland, N.Y., 1986) Vol. 73,p35.

[25] L. L. Hench in Better Ceramics Through Chemistry: MRS Symp. Proc.; Brinker, C. J., Clark, D. E., Ulrich, D. R., Eds.; (North-Holland, New York, 1984) Vol. 32, p 101.

[26] K. Tadanaga, K. Iwashita, T. Minami, and N. Tohge, J. Sol-Gel Sci. Tech. 6, 107 (1996).

[27] B. Unger, H. Jancke, M. Haehnert, and H. Stade, J. Sol-Gel Sci. Tech. 2, 51 (1994).

[28] B. Hosticka, P. M. Norris, J. Brenizer, and C. Daitch, J. Non-Cryst. Solids 225, 293 (1998).

[29] R. A. Messing, and R. A. Oppennar, Biotech. and Bioeng. 21, 49 (1979).

[30] K. Iwasaki, and N. Ueno, J. Ceram. Soc. Jpn, Int. Ed. 98, 13 (1990).

[31] K. Kajihara, K. Nakanishi, and K. Tanaka, J. Am. Ceram. Soc. 81, 2670 (1998).

[32] H. Minakuchi, K. Nakanishi, and N. Soga, Anal. Chem. 68, 3498 (1996).

[33] K. Nakanishi, N. Soga, and H. Matsuoka, J. Am. Ceram. Soc. 75, 971(1992).

[34] H. Kaji K. Nakanishi, and N. Soga, J. Sol-Gel Sci. Tech. 1, 35 (1993).

[35] J. F. T. Conroy, B. Hosticka, S. C. Davis, A. N. Smith, and P. M. Norris, Microscale Thermophys. Engin 3, 199 (1999).

[36] H. Altmann, T. Schlief, J. Gross, and J. Fricke, Ultrasonic International 91 Conf. Proc., 261 (1991).

[37] K. R. Andersson, L. S. D. Glasser, and D. N. Smith in Soluble Silicates; Falcone, J. S., Ed.; (American Chem. Soc., Washington, D. C., 1982) Vol. 194 ACS Symp. Ser., p 115.

[38] J. Gross, J. Fricke, and L. W. Hrubesh, J. Acoust. Soc. Am. 91, 2004 (1992).

[39] J. Gross, and J. Fricke, J. Non-Cryst Sol. 145, 217 (1992).

[40] J. Gross, J. Fricke, R. W. Pekala, and L. W. Hrubesh Phys. Rev. B 45, 12774 (1992).

[41] J. Gross, G. Reichenauer, and J. Fricke, J., Phys. D: Appl. Phys. 91, 1447 (1988).

[42] J. Brinker and G. Scherer in Sol-Gel Science, Academic Press, New York (1989).

[43] E. P. Plueddemann in Silane Coupling Agents, Plenum Press, New York (1991).

[44] Silicon Compounds. Register and Review 5th Ed., United Chemical Technologies, Inc., 2731 Bartram Rd., Bristol PA.

[45] B. C. Dave, B. Dunn, J. S. Valentine, and J. 1. Zink, Anal. Chem. 66, 1120A (1994).

[46] D. Avnir, S. Braun, 0. Lev, and M. Ottolenghi, Chem. Mater. 6, 1605 (1994).

[47] L. M. Ellerby, et al., Science 255, 1113 (1992).

[48] R. Campostrini, G. Carturan, R. Caniato, A. Piovan, R. Filippini, G. Innocenti, and E. M. Cappelletti, J. Sol-Gel Sci. Technol. 7, 87 (1995)

[49] M. Rietti-Shati D. Ronen, R. T. Mandelbaum. J. Sol-Gel Sci. Technol 7, 77 (1996).

What is claimed is as new and desired to be secured by Letters Patent of the united states is:

1. A method, comprising:
hydrolyzing a sol-gel precursor in water to form a sol containing an organic solvent;
removing said organic solvent from said hydrolyzed sol; and
mixing said biological material with said hydrolyzed sol after said removing step.

2. The method according to claim 1, further comprising gelling said sol to form a gel after said removing step.

3. The method according to claim 2, wherein said gelling step comprises raising a pH of said hydrolyzed sol.

4. The method according to claim 1, further comprising immobilizing said biological material.

5. The method according to claim 4, wherein said immobilizing step comprises bonding covalently said biological material to said sol.

6. The method according to claim 1, wherein said removing step comprises distilling said sol.

7. The method according to claim 1, wherein said hydrolyzing step comprises dissolving said sol-gel precursor in said water, a pH of said water being below about 4.

8. The method according to claim 1, wherein said hydrolyzing step comprises dissolving said sol-gel precursor in greater than 25 moles water per mole sol-gel precursor.

9. The method according to claim 1, wherein said hydrolyzing step comprises dissolving an alkoxy metallate in said water.

10. The method according to claim 1, further comprising mixing a dispersant into said sol.

11. The method according to claim 1, further comprising finctionalizing said sol.

12. A method comprising:
providing a sol solution having less than 29 mole % organic solvents to make said sol compatible with a biological material; and
immobilizing said biological material by mixing said biological material into said sol.

13. A method comprising:
hydrolyzing a sol-gel precursor in water to form a sol containing an organic solvent;
mixing said biological material with said sol;
mixing a sufficient amount of a dispersant into said sol to cause macropores in a gel formed by said sol.

14. A sol, comprising:
a species formed by the hydrolysis of P moles of a sol-gel precursor;
a sol solution including 71 mole % or more water and 29 mole % or less organic solvents; and
a biological material, wherein said sol solution is compatible with said biological material.

15. A sol, comprising:
a species formed by the hydrolysis of P moles of a sol-gel precursor;
W moles of water;
a sufficient amount of a dispersant to cause macropores in a gel formed by said sol;
and
a biological material.

16. The sol according to claim 15, wherein said dispersant comprises a water-soluble polymer.

17. The sol according to claim 15, wherein a hydrolysis ratio of WP is greater than 25:1.

18. The sol according to claim 15, wherein said sol-gel precursor comprises an alkoxy metallate.

19. The sol according to claim 18, wherein said alkoxy metallate comprises an alkoxy silicate.

20. The sol according to claim 15, further comprising a means for functionalizing a gel formed by condensation of said hydrolyzed species.

21. The sol according to claim 15, wherein said biological material comprises a cell.

22. The sol according to claim 21, further comprising a nutrient supply configured to support said biological cell.

23. The sol according to claim 15, wherein said sol comprises a sol solution, said W moles of water forming at least 71 mole % of said sol solution.

24. The sol according to claim 15, wherein said organic solvents comprise an organic by-product arising from a production of said sol-gel precursor.

25. The sol according to claim 15, wherein a hydrolysis ratio of W:P is greater than 100:1.
